(19) United States
(12) Patent Application Publication
(10) Pub. No.: US 2005/0048165 A1
(43) Pub. Date: Mar. 3, 2005

(54) Process for the preparation of Chapathi dough with reduced phytic acid level

(76) Inventors: Sadanandan Bindu, Bangalore (IN); Mandyam Chakravarathy Varadaraj, Mysore (IN)

Correspondence Address: NATH & ASSOCIATES, 1030 15th STREET, NW, 6TH FLOOR, WASHINGTON, DC, 20005, US

(21) Appl. No.: 10/813,574

(22) Filed: Mar. 31, 2004

Related U.S. Application Data
(60) Provisional application No. 60/458,416, filed on Mar. 31, 2003.

(51) Int. Cl. A23L 1/28; C12N 1/18

(52) U.S. Cl. 426/28; 435/254.22

(57) ABSTRACT

The present invention relates to a process for the preparation of Chapathi dough with reduced phytic acid using a mutant of Candida versatilis.

### FIELD OF THE PRESENT INVENTION

The following specification particularly describes the nature of the invention and the manner in which it is to be performed. The present invention relates to a process for the preparation of Chapathi dough with reduced phytic acid by using two mutants of yeast.

### BACKGROUND AND PRIOR ART REFERENCES

Wheat is the most commonly consumed cereal in the world. Whole meal bread of wheat is a staple food in many countries and is an important source of protein and minerals. The high phytic acid content in whole wheat flour products render a low availability of nutrients, especially the minerals. Chapathi, a traditional unleavened flat bread (pancake) prepared from whole wheat flour is the staple food of north India as well as in Pakistan, Iran and other Middle East Asian countries. In recent times, Chapathi is also consumed by a larger section of human population in other regions of India, more so from the point of health benefits. This product is almost synonymous with unleavened flat bread consumed elsewhere in the world like African and European countries.

In whole wheat flour used in Chapathi preparation is not refined and usually contains bran. Hence, phytic acid is found in this traditional food item. As minerals, vitamins and dietary fibre are bound together with the phytate, a mechanical removal of phytate by removing the outer grain layers will lower the nutritive as well as the organoleptic qualities of the resultant product. However, to retain these qualities, the phytate needs to be removed enzymatically. Phytase is the enzyme that hydrolyzes phytate.

Phytase is a phosphomonoesterase capable of hydrolyzing myo-inositol hexakisphosphate to yield inorganic orthophosphate and a series of lower phosphoric esters of myo-inositol and ultimately in some cases, free myo-inositol. Phytase is naturally present along with phytate in wheat grains, but usually gets inactivated due to long term storage of wheat as well as during processing of wheat into its products. Chapathi being a non-fermented food product, there is no scope for the activation of grain phytases.

Reference may be made to the work of Han and Wilfred (1988), wherein phytase from Aspergillus ficuum was used to reduce 67-85% of phytate soy bean meal and cotton seed meal. Reference may be made to the work of Nair et al. (1991), wherein an apparent 10% increase in protein content of the canola meal was noted as a result of the growth of Aspergillus ficuum.

Reference may be made to the studies of Segueilla et al. (1993), wherein phytase from Schwannionmyces castelli was used to make bioavailable of phytate phosphorus in many of the livestock feeds. Reference may be made to the work of Liu et al. (1997), wherein supplementation of microbial phytase significantly improves phosphorus availability in corn-soy bean meal diets as feed supplements. Reference may be also be made to the work of Han et al. (1999), wherein the recombinant phytase gene in Saccharomyces cerevisiae was effective in hydrolysing phytate phosphorus from corn or soy bean meal. Reference may be made to the studies of Matsui et al. (2000), wherein the phytase produced by Aspergillus niger was found to be more effective as against the yeast phytase.

Reference may be made to the studies of Simon et al. (1990), Jongbloed and Kemme (1990) and Jongbloed et al. (1992), wherein addition of enzyme phytase to animal feeds resulted in levels of performance equal to or better than those attained by supplementing inorganic phosphate. Reference may be made to the work of Lei et al. (1993), wherein it was demonstrated that addition of phytase to the diets of weanling pigs improved the bioavailability of zinc and phytate phosphorus. Reference may be made to the studies of Kornegay et al. (1996), wherein it was observed that when graded levels of microbial phytase was added to maize-soy bean meal-based diets, bioavailability of minerals increased leading to increase in body weight gain, feed intake, calcium retention and total amount of phosphorus retained.

Microbial fermentation has shown to markedly increase hydrolysis native phytic acid. Reference may be made to the work of Navert and Sandstrom (1985), wherein there was reduction in phytic acid content of bread containing bran after 2 h of leavening with yeast. Owing to high phytic acid content, the bioavailability of minerals from whole wheat bread is low. Reference may be made to the studies of Turk et al. (1996), wherein reduction in the levels of phytate was observed during whole-meal bread making due to the effect of yeast and wheat phytases.

Reference may be made to the work of Gupta et al. (1990), wherein fermentation of freshly ground wheat flour-butter milk mixture during Rabadi preparation resulted in reduction of phytic acid levels. Reference can be made to the work of Agte and Joshi (1987), wherein traditional food processing methods such as soaking and cooking can activate native phytases resulting in hydrolysis of phytate in wheat and millets. Reference may be made to the work of Turk et al. (1996), wherein the knowledge of how to optimize phytase activity during bread making and food processing can be used to produce foods with a higher mineral bioavailability.

Microbial cells can be permeabilized without lysis of cells or destruction of the whole inner organization. Permeabilized cells have the advantage of that the range of measurable enzymes is much bigger and enzymes regenerating energy carriers as NADH and ATP are available in the same organism.

The drawback of all these methods is the use of microbial phytase in plant-based meals, primarily used as animal feeds. Besides, the process of phytic acid reduction was the result of microbial fermentation. The same principle does not hold any validity in foods devoid of fermentation. Further, the suitability of microbial phytase in reducing phytic acid in traditional cereal-based non-fermented foods has not been attempted in any of the earlier works/studies. At the same time there have been no patents taken in the area of using yeast phytase as a source to reduce phytic acid in traditional unfermented foods.

### OBJECTS OF THE PRESENT INVENTION

The main object of the present invention is to provide a process for the preparation of Chapathi dough with reduced phytic acid which obviates the drawbacks detailed above.

Another object of the present invention is to use potent yeast culture positive for phytase activity.

Still another object of the invention is to optimize conditions for effective permeabilization of yeast cells with higher phytase activity.

Yet another object of the invention is to utilize the permeabilized yeast cells in reducing the phytic acid level in Chapathi dough

### SUMMARY OF THE PRESENT INVENTION

The present invention relates to a process for the preparation of Chapathi dough with reduced phytic acid level by using two mutants of yeast.

### DETAILED DESCRIPTION OF THE PRESENT INVENTION

The present invention relates to a process for the preparation of Chapathi dough with reduced phytic acid.

Accordingly, the present invention provides a process for the preparation of Chapathi dough with 10-45% reduction in phytic acid level, using mutated strain of a yeast Candida versatilis accession number having phytase activity ranging between 140-170 U/g, the said process comprise steps of:
a) adding water to wheat flour in a ratio of 1:0.5-1:0.6 and NaCl of concentration ranging from 0.8-1.2% to obtain Chapathi dough;
b) mixing the permeabilized yeast mutants with the Chapathi dough of step (a), and
c) storing of Chapathi dough at a temperature ranging between 10-26° C. for a period of 30 min to 24 h and thereby obtaining the Chapathi dough with 10-45% reduction in the level of phytic acid.

In another embodiment of the present invention a mutated strain of yeast Candida versatilis deposited in international depository with an accession number Still in another embodiment of the present invention the mutation yeast strain was performed by Ethyl methane sulphonate (EMS).

Yet in another embodiment of the present invention the highest mutation frequency was achieved with a concentration of about 30 mg/ml of EMS in reaction mixture with time duration of about 150 minutes and rotation per minute (RPM) of about 150 at about room temperature.

Yet in another embodiment of the present invention wherein permeabilizing the mutant is carried out by freezing and thawing.

Further in another embodiment of the present the freezing and thawing was performed by ice-salt mixture/liquid nitrogen and tap water respectively.

In one more embodiment of the present invention the highest permeabilization of cells of mutants were obtained by liquid nitrogen freezing for 20 seconds and thawing under tap water at about 26° C. for about 40 seconds and repeating the process for about 4 cycles.

Still in another embodiment of the present invention the highest permeabilization of cells of mutants were obtained by ice-salt freezing for about 10 minutes and thawing under tap water at about 26° C. for about 40 seconds and repeating the process for about 20 cycles.

Yet in another embodiment of the present invention the maximum phytase activity is obtained in the liquid nitrogen freeze-thawed permeabilized mutants.

In one more embodiment of the present invention a reduction of 45% in the level of phytic acid in Chapathi dough is achieved in about 24 h at about 10° C.

Still in one more embodiment of the present invention a mutated strain of a yeast Candida versatilis deposited in international depository number and having the accession number ______.

Yet in another embodiment of the present invention the mutation was performed by UV radiation.

In one more embodiment of the present invention the highest mutation frequency was achieved with about 2 minutes of UV irradiation from about 30W UV lamp from a distance of about 25 cms.

Still in another embodiment of the present invention a process for the preparation of Chapathi dough with 10-45% reduction in phytic acid level using mutated a yeast Candida versatilis accession number ______ having phytase activity ranging between 140-170 U/g, where the said process comprising the steps of:
a) adding water to wheat flour in a ratio of 1:0.5-1:0.6 and NaCl of concentration ranging from 0.8-1.2% to obtain Chapathi dough;
b) mixing the permeabilized yeast mutants with the Chapathi dough of step (a), and
c) storing of Chapathi dough a temperature ranging between 10-26° C. for a period of 30 min to 24 h and thereby obtaining the Chapathi dough with 10-45% reduction in the level of phytic acid.

Further in another embodiment of the present invention the permeabilizing the mutant of Candida versatilis is carried out by freezing and thawing.

In another embodiment of the present invention, wherein the freezing and thawing was performed by ice-salt mixture/liquid nitrogen and tap water respectively.

Further in another embodiment of the present invention the highest permeabilization of cells of mutants were obtained by ice-salt freezing for about 10 minutes and thawing under tap water at about 26° C. for about 40 seconds and repeating the process for about 20 cycles.

Still in another embodiment of the present invention the maximum phytase activity is obtained in the liquid nitrogen freeze-thawed permeabilized mutants.

In an embodiment of the present invention, the potent culture is a mutant EMY 505 of Candida versatilis CFR 505.

The process relates to a process for the preparation of Chapathi dough with reduced phytic acid. The potent yeast culture selected in bringing about this desirable effect was a native food isolate of Candida versatilis CFR 505. Mutagenesis with ethyl methanesulphonate and UV irradiation resulted in desirable mutants—EMY 505 and UVY 505 with higher phytase activity. The cells were subjected to permeabilization by freeze-thawing cycles with liquid nitrogen and ice-salt mixture. The permeabilized cells of the mutants EMY 505 and UVY 505 obtained by freeze-thawing in liquid nitrogen showed an increase of 197 and 170 U/g, cells, respectively, relative to the permeabilized cells of wild type of Candida versatilis CFR 505. A significant reduction in phytic acid level to the extent of 45% was obtained in Chapathi dough in 24 h at 110° C. storage, when freeze-thawed permeabilized cells of EMY 505 mutant of Candida versatilis was mixed during the preparation of Chapathi dough. The process establishes the biotechnological approach focusing phytase active yeast cultures in improving the nutritional status of a traditional cereal-based non-fermented food item.

The novelty of the process lies in the ability to reduce inherent anti-nutritional factor like phytic acid in traditional wheat flour based non-fermented food to a reasonable extent using permeabilized cells of phytase positive potent isolate of Candida versatilis. As against the more common use of yeast fermentation for deriving phytic acid reduction, there has been no attempts to evolve means to overcome anti-nutritional factors in plant-based foods, wherein fermentable is an undesirable attribute. The present process provides a biotechnological approach towards improving the nutritional status of Chapathi dough.

### EXAMPLES

The following examples are given by way of illustrations of the present invention and therefore should not be construed to limit the scope of the present invention.

### Example-1

In the entire microbiological work, sterilization is achieved by autoclaving different growth and plating media, glassware and other solutions for 20 min at 121° C.

Samples of idli batter in 100 g from different habitats (home-made, hotels and catering centre) were collected in sterile glass beakers. Prior to use, the glass beakers were washed well, air dried, mouth covered with aluminium foil and autoclaved at 121° C. for 20 mm. Sample aliquots in 11 g each were added into 99 ml of sterile 0.85% saline to get an initial 10⁻¹ dilution. From this initial sample dilution, subsequent serial dilutions were prepared in 9 ml amounts of sterile 0.85% saline. Aliquots of appropriate sample dilutions in 1 ml were pour plated with conventional nutrient medium such as potato dextrose agar (PDA) for the enumeration and isolation of predominant genera of yeasts. Poured plates of PDA were incubated at 30° C. for 72 h.

On the basis of colony morphology, well separated colonies of yeasts formed on PDA plates were selected at random and transferred aseptically onto PDA slants. Inoculated slants were incubated at 30° C. for 72 h. Incubated tubes showing good growth were stored at 6° C. with sub-culturing in PDA slants at regular intervals of 15 days. All the selected natural isolates were maintained in 2 sets. Isolated cultures were characterized by morphological, cultural and biochemical tests. The tests included were assimilation of carbon sources like cellobiose, erythritol, galactose, maltose, mannitol, melibiose, raffinose, trehalose and xylose; assimilation of nitrogen sources; growth at 37° C.; pellicle formation in broth; resistance to 0.01% cycloheximide; glucose fermentation; splitting of urea.

The isolates identified were those of Candida versatilis. The characteristics of the identified yeast culture are presented in Table 1. The identified isolate is deposited at Central Food Technological Research Institute culture collection centre, Mysore and designated as Candida versatilis CFR 505.

TABLE 1
Cultural and biochemical characteristics of natural isolate of yeast

| Characteristics | Yeast isolate |
|---|---|
| Growth at 37° C. | + |
| Pellicle formation | − |
| Growth in 0.01% cycloheximide | + |
| Glucose fermentation | + |
| Splitting of urea | + |
| Nitrogen assimilation | + |
| Carbon assimilation | |
| Cellobiose | − |
| Erythritol | − |
| Galactose | + |
| Maltose | + |
| Mannitol | − |
| Melibiose | − |
| Raffinose | + |
| Trehalose | − |
| Xylose | − |
| Identified species | Candida versatilis |

+, positive for reaction;
−, negative for reaction

### Example-2

Mutants of Candida versatilis CFR 505 was obtained by mutagenesis with ethyl methane sulphonate (EMS) and UV irradiation. Mutagenesis was performed according to the method of Lindegren et al. (1965) with slight modification. The reaction mixture contained 8 ml quantity of sterile 0.1 M acetate buffer into which was added 1 ml of the cell suspension containing 2×10⁹ CFU of the organism and 1 ml aliquot of the individual concentration of EMS, which ranged between 6 and 60 mg/ml. The experimental flasks with the reaction mixture were gently agitated in an orbital shaker for 150 min at 150 rpm at ambient temperature. Subsequently, the cells were washed 4 times with 0.1 M acetate buffer and the same were enumerated for viable count and mutation assessment by surface plating in duplicate on pre-poured plates of potato dextrose agar and incubated for 72 h at 30° C. Colonies of surviving cells appearing on the medium and showing any marked morphological differences from that of wild type were recorded and isolated.

Mutagenesis by UV irradiation was carried out following the method of Chelius and Wodzinski (1994) with slight modification The UV irradiation source was a 30 W lamp (Sylvania) emitting radiation at 254 nm. One ml of cell suspension containing 2×10⁹ CFU was added to 9 ml of 0.1 M acetate buffer of pH 5.0 taken in a sterile glass petri plate (100×17 mm). The cell suspension was irradiated from a distance of 25 cm, while stirring for up to 10 min. After irradiation, the surviving cells were appropriately diluted and 0.1 ml aliquot was surface plated in duplicate on pre-poured plates of potato dextrose agar and incubated for 72 h at 30° C. Results were recorded as described under EMS treatment. After UV treatment, all manipulations were done in subdued light.

The wild and mutants of Candida versatilis CFR 505 were assayed for phytase activity. Mutants that have survived EMS and UV treatments were qualitatively screened for phytase production through point inoculations of the individual mutants on phytase screening agar medium by the method described by Howson and Davis (1983). After 72 h of incubation at 30° C., the ratio of colony diameter to the distance of agar clearing from the colony edge was measured and used to estimate phytase production.

Point inoculations of wild type colonies served as positive control. Cell-associated phytase activity in the mutants and wild type was determined using cells obtained from 20 h-old culture broth. Aliquots of cells in 2 g each, were then homogenized in a pre-chilled pestle and mortar using glass beads of 212-300μ (Sigma, St. Louis, Mo., USA) with protease inhibitor cocktail of 2 mmol/l PMSF, EDTA and 2-mercaptoethanol in 10% glycerol. The homogenate was centrifuged at 12000 g for 15 min at 4° C. and the resultant cell-free extract was used for the assay. Phytase activity was determined by measuring the liberated inorganic phosphate from phytate according to the method described by Ullah and Gibson (1987). The assay was performed at a pH level of 5.0 for 35 min at 50° C. in a thermostatically controlled water bath. One unit (U) of phytase activity is defined as 1 μmol inorganic phosphorus produced per min per ml at pH 5.0 and 50° C.

For the strain of Candida versatilis CFR 505, the highest mutation frequency for phytase production was at a concentration of 30 mg/ml of EMS and the mutant was designated as EMY-505. A complete inhibition occurred with a concentration of 60 mg/ml of EMS. In a UV exposure time of 8 min, 100% kill was observed with Candida versatilis CFR 505. A UV exposure of 2 min resulted in the highest mutation frequency and these mutants were termed as UVY-505. Both the EMS and UV mutants showed an appreciable difference in their colony morphology, relative to the wild type.

Point inoculation of wild type and mutants (EMY 505 and UVY 505) in phytase screening agar medium failed to show any extracellular phytase activity. Cell-associated phytase activity was found to be higher in mutants with a total activity of 167.87 and 142.52 U/g, cells, respectively with EMY 505 and UVY 505. In the mutants of Candida versatilis EMY-505 and UVY-505, the increase was 1.76 and 1.63-fold, respectively.

### Example-3

Cells of wild and mutants (EMY 505 and UVY 505) of Candida versatilis CFR 505 were prepared individually. Aliquots of sterile 250 ml of potato dextrose broth taken in 1000 ml Erlenmeyer conical flask was inoculated with a speck of the mutants, individually and incubated for 18 h at 30° C. in an Orbital Shaker at a rpm of 200. Cells were harvested from the incubated culture broth by centrifugation at 1100 g for 10 min at 4° C. The resultant supernatant was discarded and the cell pellet washed twice with 0.1 M sodium acetate buffer of pH 5.0. These cells were subjected to permeabilization by freezing and thawing.

The wet cells of the wild and two mutants, individually in aliquots of 1 g quantity was suspended in 10 ml of sodium acetate buffer. This suspension was frozen in freezing mixture (ice-salt) for 10 min and then thawed at 26° C. for 10 min under running tap water. This process of freezing and thawing was repeated for up to 25 cycles. The resultant permeabilized cells were washed with sodium acetate buffer and used for assay of phytase activity.

In another set of experimental trials, freezing of the wet cells of the wild and two mutants, individually was carried out in liquid nitrogen for 20 seconds, followed by thawing at 26° C. for 40 seconds under running water. The resultant permeabilized cells were washed with sodium acetate buffer and used for assay of phytase activity.

Aliquots of 0.1 g of wet cells of wild and mutants of Candida versatilis CFR 505, individually, were suspended in 1.5 ml of 0.2 mM acetate buffer of pH 5.0 and 0.5 ml of 5 mM sodium phytate. After incubation at 50° C. for 35 min, the reaction was stopped by adding 2 ml of 10% trichloroacetic acid. The liberated inorganic phosphate (Pi) was determined according to the method of Heinonen and Lahti (1981). One unit of phytase activity was defined as one micromole per litre of inorganic phosphate released from sodium phytate per minute under the assay conditions.

Using ice-salt freezing and thawing, 20 cycles were required to obtain maximal phytase activity in the wild strain of Candida versatilis CFR 505. In the penneabilized cells, there was an increase of 35 U/g, cells, relative to the non-permeabilized cells. The mutants, EMY 505 and UVY 505 recorded an increase of 112 and 99 U/g, cells, respectively, as against the permeabilized cells of wild type. In the case of freezing and thawing using liquid nitrogen, 4 cycles resulted in an increased phytase activity of 90 U/g, cells in wild strain of Candida versatilis CFR 505. The permeabilized cells of EMY 505 and UVY 505 showed an increase of 197 and 170 U/g, cells, respectively, relative to the wild type.

### Example-4

Aliquots of whole wheat flour in 100 g quantities each were taken in clean SS vessels to which were added 2×10⁸ CFU of freeze-thawed permeabilized cells of wild and two mutants (EMY 505 and UVY 505) of Candida versatilis CFR 505, individually. The added cells were mixed uniformly with the whole wheat flour using a sterile flat spoon. To the individual mixture of wheat flour and respective permeabilized yeast cells were added 45 ml of sterile distilled water, 1 g table salt and kneaded well into dough. The prepared Chapathi dough was divided into two equal parts, with one portion being kept at 26° C. for 30 min and the other portion for 24 h at 10° C. Phytic acid content was determined in 1 g quantity of the above two stored samples of Chapathi dough. The method of extraction, purification and Spectrophotometric determination of phytic acid was performed according to the published method of Fruhbeck et al. (1995).

The use of freeze-thawed permeabilized cells of the wild strain of Candida versatilis CFR 505 failed to reduce the native phytic acid level in the Chapathi dough. In Chapathi dough added with freeze-thawed permeabilized cells of the mutant EMY 505, there was a significant reduction in phytic acid level. In a storage period of 30 min at 26° C., the permeabilized cells derived by freeze-thawing using liquid nitrogen were able to bring about a 39.9% reduction in phytic acid level. The reduction was 45.1% in 24 h at 10° C. (Table 2). The reduction achieved with freeze-thawed cells of mutant UVY 505 was lower as compared with the mutant of EMY 505 (Table 3). There was no reduction in phytic acid during storage of Chapathi dough (control) as well as in dough added with non-permeabilized cells of wild and mutants.

TABLE 2
Effect of freeze-thawed permeabilized
cells of mutant EMY 505 of Candida versatilis
CFR 505 on phytic acid level in Chapathi dough

| | Storage Condition | |
|---|---|---|
| | 30 min at 26° C. | 24 h at 10° C. |
| Sample type | (Phytic acid content mg/g, dough) | |
| Chapathi dough (control) | 13.5^{a} | 13.45^{a} |
| Chapathi dough + non-permeabilized | 13.31^{a} | 13.22^{a} |
| cells | | |
| Chapathi dough + permeabilized | 10.95^{b} | 19.19^{b} |
| cells (ice-salt mixture) | (18.9%) | (24.2%) |
| Chapathi dough + permeabilized | 8.9^{c} | 7.37^{c} |
| cells (liquid nitrogen) | (39.9%) | (45.1%) |

Mean values with different letters in each column indicate significant difference (p < 0.05) based on Duncan's new multiple range test
Figures in parentheses indicate percent degradation of phytic acid

TABLE 3
Effect of freeze-thawed permeabilized
cells of mutant UVY 505 of Candida versatilis
CFR 505 on phytic acid level in Chapathi dough

| | Storage condition | |
|---|---|---|
| | 30 min at 26° C. | 24 h at 10° C. |
| Sample type | (Phytic acid content mg/g, dough) | |
| Chapathi dough (control) | 13.5^{a} | 13.45^{a} |
| Chapathi dough + non-permeabilized | 13.40^{a} | 13.45^{a} |
| cells | | |
| Chapathi dough + permeabilized | 11.94^{b} | 11.26^{b} |
| cells (ice-salt mixture) | (11.6%) | (16.3%) |
| Chapathi dough + permeabilized | 10.86b | 10.18^{b} |
| cells (liquid nitrogen) | (20.0%) | (24.32%) |

Mean values with different letters in each column indicate significant difference (p < 0.05) based on Duncan's new multiple range test
Figures in parentheses indicate percent degradation of phytic acid

The main advantages of the present invention are:

1. A biotechnological approach to reduce anti-nutritional factor like phytic acid in Chapathi, a traditional food product of India.

2. Selection of a potent yeast culture with phytase activity.

3. Simpler methods of mutation and permeabilization to achieve higher phytase activity.

1) a mutated strain of a yeast Candida versatilis deposited in international depository with an accession number ______.

2) the mutated yeast strain as claimed in claim 1, wherein the mutation was performed by ethyl methane sulphonate (EMS).

3) The mutated yeast strain as claimed in claim no 2, wherein the highest mutation frequency was achieved with a concentration of about 30 mg/ml of EMS in reaction mixture with time duration of about 150 minutes and rotation per minute (RPM) of about 150 at about room temperature.

4) A process for the preparation of Chapathi dough with 10-45% reduction in phytic acid level, using mutated strain of a yeast Candida versatilis accession number ______ having phytase activity ranging between 140-170 U/g, the said process comprise steps of:
a) adding water to wheat flour in a ratio of 1:0.5-1:0.6 and NaCl of concentration ranging from 0.8-1.2% to obtain Chapathi dough;
b) mixing the permeabilized yeast mutants with the Chapathi dough of step (a), and
c) storing of Chapathi dough at a temperature ranging between 10-26° C. for a period of 30 min to 24 h and thereby obtaining the Chapathi dough with 10-45% reduction in the level of phytic acid.

5) A process as claimed in claim 4, wherein permeabilizing the mutant by freezing and thawing.

6) A process as claimed in claim 5, wherein the freezing and thawing was performed by ice-salt mixture/liquid nitrogen and tap water respectively.

7) A process as claimed in claim 6, wherein the highest permeabilization of cells of mutants were obtained by liquid nitrogen freezing for 20 seconds and thawing under tap water at about 26° C. for about 40 seconds and repeating the process for about 4 cycles.

8) A process as claimed in claim 5, wherein the highest permeabilization of cells of mutants were obtained by ice-salt freezing for about 10 minutes and thawing under tap water at about 26° C. for about 40 seconds and repeating the process for about 20 cycles.

9) A process as claimed in claims 6, wherein maximum phytase activity is obtained in the liquid nitrogen freeze-thawed permeabilized mutants.

10) A process as claimed in claim 4, wherein a reduction of 45% in the level of phytic acid in Chapathi dough is achieved in about 24 h at about 10° C.

11) A mutated strain of a yeast Candida versatilis deposited in international depository number and having the accession number ______.

12) A mutated yeast strain as claimed in claim 11, wherein the mutation was performed by UV radiation.

13) The mutated yeast strain as claimed in claim no 12, wherein the highest mutation frequency was achieved with about 2 minutes of UV irradiation from about 30W UV lamp from a distance of about 25 cms.

14) A process for the preparation of Chapathi dough with 10-45% reduction in phytic acid level using mutated a yeast Candida versatilis accession number having phytase activity ranging between 140-170 U/g, where the said process comprising the steps of:
a) adding water to wheat flour in a ratio of 1:0.5-1:0.6 and NaCl of concentration ranging from 0.8-1.2% to obtain Chapathi dough;
b) mixing the permeabilized yeast mutants with the Chapathi dough of step (a), and
c) storing of Chapathi dough a temperature ranging between 10-26° C. for a period of 30 min to 24 h and thereby obtaining the Chapathi dough with 10-45% reduction in the level of phytic acid.

15) The process as claimed in claim 14, wherein the mutation of Candida versatilis 505 was performed by UV radiation.

16) The mutated yeast strain as claimed in claim no 15, wherein the optimized parameters of UV radiation to obtain highest mutation frequency was about 2 minutes of irradiation from about 30W UV lamp from a distance of about 25 cms.

17) A process as claimed in claim 14, wherein the permeabilizing the mutant of Candida versatilis by freezing and thawing.

18) A process as claimed in claim 16, wherein the freezing and thawing was performed by ice-salt mixture/liquid nitrogen and tap water respectively.

19) A process as claimed in claim 17, wherein the highest permeabilization of cells of mutants were obtained by liquid nitrogen freezing for 20 seconds and thawing under tap water at about 26° C. for about 40 seconds and repeating the process for about 4 cycles.

20) A process as claimed in claim 17, wherein the highest permeabilization of cells of mutants were obtained by ice-salt freezing for about 10 minutes and thawing under tap water at about 26° C. for about 40 seconds and repeating the process for about 20 cycles.

21) A process as claimed in claims 17, wherein maximum phytase activity is obtained in the liquid nitrogen freeze-thawed permeabilized mutants.
